(19) United States
(12) Patent Application Publication
(10) Pub. No.: US 2008/0300607 A1
(43) Pub. Date: Dec. 4, 2008

(54) Apparatus and Method for Introducing Implants

(75) Inventors: Noah Meade, Atlanta, GA (US); Doug Evans, Snellville, GA (US)

Correspondence Address: Rutan & Tucker, LLP., 611 ANTON BLVD, SUITE 1400, COSTA MESA, CA, 92626, US

(73) Assignee: C.R. BARD, Inc., Murray Hill, NJ (US)

(21) Appl. No.: 12/159,589

(22) PCT Filed: Dec. 28, 2006
(86) PCT No.: PCT/US2006/062639

§ 371 Date: Aug. 15, 2008

Related U.S. Application Data
(60) Provisional application No. 60/754,265, filed on Dec. 28, 2005.
(60) Provisional application No. 60/810,065, filed on Jun. 1, 2006.

(30) Foreign Application Priority Data
Aug. 3, 2006 (US) PCT US2006/030369
Aug. 3, 2006 (US) PCTUS2006/030370
Aug. 3, 2006 (US) PCTUS2006/030581

(51) Int. Cl. A61B 17/42

(52) U.S. Cl. 606/119; 600/37; 128/898; 606/185

(57) ABSTRACT

In one embodiment, an introducer includes a handle, a needle extending from the handle, the needle having a distal end and defining an inner lumen, the needle further having an opening that provides access to the inner lumen, a snare having an implant coupling element, the snare being positioned within the inner lumen of the needle, the snare being extendable from the needle opening to an extended position in which the implant coupling element is positioned outside of the inner lumen and retractable to a retracted position in which at least a portion of the implant coupling element is positioned within the inner lumen, and a snare extension mechanism provided on the handle, the extension mechanism being configured to alternately extend and retract the snare.

### CROSS-REFERENCE TO RELATED APPLICATIONS

This application is related to copending U.S. Provisional Patent Application No. 60/754,265, filed Dec. 28, 2005, and POT Application No. PCT/US2006/030369, filed Aug. 3, 2006, and PCT Application No PCT/US2006/03051, filed Aug. 3, 2006, and PCT application No. PCT/US2006/030370, filed Aug. 3, 2006, and U.S. Provisional Patent Application No. 60/810,065, filed Jun. 1, 2006, all of which are entirely incorporated herein by reference in their entirety.

### BACKGROUND

Surgical devices referred to as “introducers” are often used to implant or “introduce” implantable devices within the body. For example, such introducers can be used to position within the pelvis mesh implants intended for treating urinary incontinence or performing prolapse repair.

Positioning an implant within the human body, such as within the pelvis, can be challenging due to the anatomy of the body and the placement of the implant that may be required to treat a given ailment. For instance, the treatment of rectocele, a condition in which the rectum encroaches on the vagina, may require accessing the vaginal vault from a position deep within the pelvis so as to form a passage in which a portion, such as an anchoring arm, of the implant can be placed. Formation of such a passage typically requires a relatively high degree of skill.

Further complicating implantation of a rectocele implant, or other such pelvic implant, is the need to draw the implant into the body and through the formed passage. In present techniques, a needle is passed through a pelvic incision, through the soft tissue of the pelvis, into the vagina, down through the vagina, and out the vaginal introits to enable the implant to be connected to the needle so that the needle may then be withdrawn with the implant in tow to position the implant within the formed passage. Given the configuration and dimensions of the human pelvis and its organs, it can be difficult to navigate a needle through such a tortuous path without causing damage to or otherwise disrupting the tissues of the pelvis, such as the pelvic floor muscles.

### BRIEF DESCRIPTION OF THE DRAWINGS

The disclosed systems can be better understood with reference to the following drawings. The components in the drawings are not necessarily to scale.

FIG. 1 illustrates a first embodiment of an introducer system.

FIG. 2 is a perspective view of an introducer shown in FIG. 1.

FIG. 3 illustrates passage of a snare shown in FIG. 1 through the introducer shown in FIG. 2.

FIG. 4 illustrates a second embodiment of an introducer system.

FIG. 5 is a perspective view of an introducer shown in FIG. 4.

FIG. 6 illustrates passage of a snare shown in FIG. 1 through the introducer shown in FIG. 5.

FIGS. 7A-7K illustrate steps performed in a first embodiment of a method for implanting a pelvic implant within the body.

FIGS. 8A and 8B illustrate steps performed in a second embodiment of a method for implanting a pelvic implant within the body.

FIG. 9 is a side view of an alternative embodiment of a snare that can be used in an introducer system.

FIG. 10 is a partial front view of the snare of FIG. 9, illustrating an implant coupling element of the introducer.

FIG. 11 is a perspective view of an alternative embodiment of an introducer that can be used in an introducer system.

FIG. 12 illustrates securing of a snare to the introducer shown in FIG. 11 using a cleat of the snare.

FIG. 13 is a side view of a further alternative embodiment of an introducer.

FIG. 14 is a perspective view of the introducer of FIG. 13, illustrating a retracted state of an internal snare of the introducer.

FIG. 15 is a perspective view of the introducer of FIG. 13, illustrating an extended state of an internal snare of the introducer.

FIG. 16 is a first exploded perspective view of the introducer of FIG. 13.

FIG. 17 is a second exploded perspective view of the introducer of FIG. 13.

FIG. 18 is a detail view of components of the introducer of FIG. 13, illustrating coupling of a slide element and an internal snare to an internal carrier element.

FIG. 19 is a side view of yet another alternative embodiment of an introducer.

### DETAILED DESCRIPTION

As described above, it can be difficult to position an implant within the body. That may particularly be the case in relation to positioning a pelvic implant intended for use in treating incontinence or performing prolapse repair. For example, as described above, a surgeon may need to access a point deep within the pelvis, such as the vaginal vault, with an introducer and connect an implant to the introducer at a point outside of the body to enable the implant to be drawn through a passage formed in the soft tissues of the pelvis by the introducer. It is difficult to perform such a procedure with current introducers given that the introducer must traverse a tortuous path to extend outside of the body from a point deep within the pelvis.

Disclosed herein are systems, methods, and apparatuses that simplify implantation of an implantable device, such as a pelvic implant. In some embodiments, an introducer system comprises a snare that can be extended from a tip of an introducer needle to a position outside of the body when the tip is positioned at a point within the body, such as within vagina. In such a case, an implant can be coupled to the extended snare and the snare can then be retracted to pull the implant through the body and at least to the tip of the introducer needle. In some embodiments, both the snare and the implant can further be drawn through the introducer needle such that the implant traverses the passage formed by the introducer needle without direct contact with the tissues of the passage, thereby reducing irritation to the soft tissues in which the passage is formed.

In the following, various embodiments of systems, methods, and apparatuses are described in detail. Although specific embodiments are presented, those embodiments are mere exemplary implementations of the disclosed systems, methods, and apparatuses and it is noted that other embodiments are possible. All such embodiments are intended to fall within the scope of this disclosure.

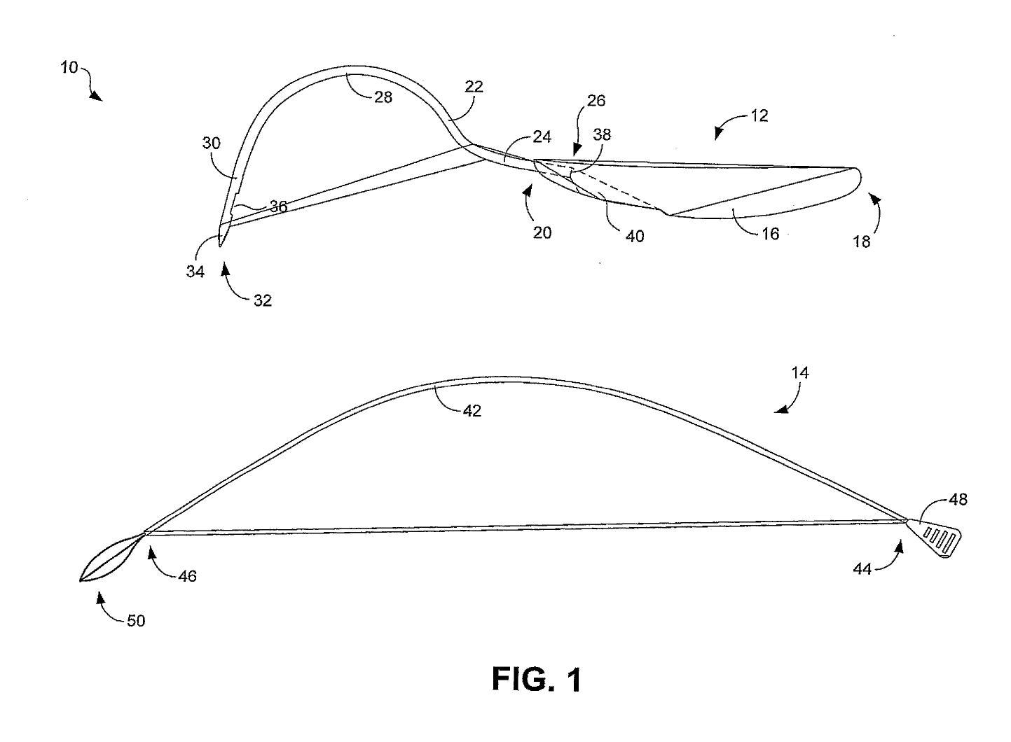

FIG. 1 illustrates a first embodiment of an introducer system 10. The introducer system 10 is well suited for use in performing prolapse repair, such as anterior prolapse repair and treating cystocele. As indicated in FIG. 1, the system 10 includes an introducer 12 and a snare 14. The introducer 12 comprises a handle 16 that includes a proximal end 18 and a distal end 20. The handle 16 is generally sized and shaped to fit within a surgeon's hand and, as depicted in FIG. 1, can be curved to facilitate firm gripping.

A needle 22 extends from the distal end 20 of the handle 16. As shown in FIG. 1, at least a portion of the needle 22 is curved. In the embodiment of FIG. 1, the needle 22 comprises a first generally straight portion 24 adjacent its proximal end 26, a curved portion 28 in a central region, and a second generally straight portion 30 adjacent its distal end 32. Formed at the distal end 32 is a blunt point or tip 34 that is configured to dissect soft tissue as the needle 22 is passed through the body.

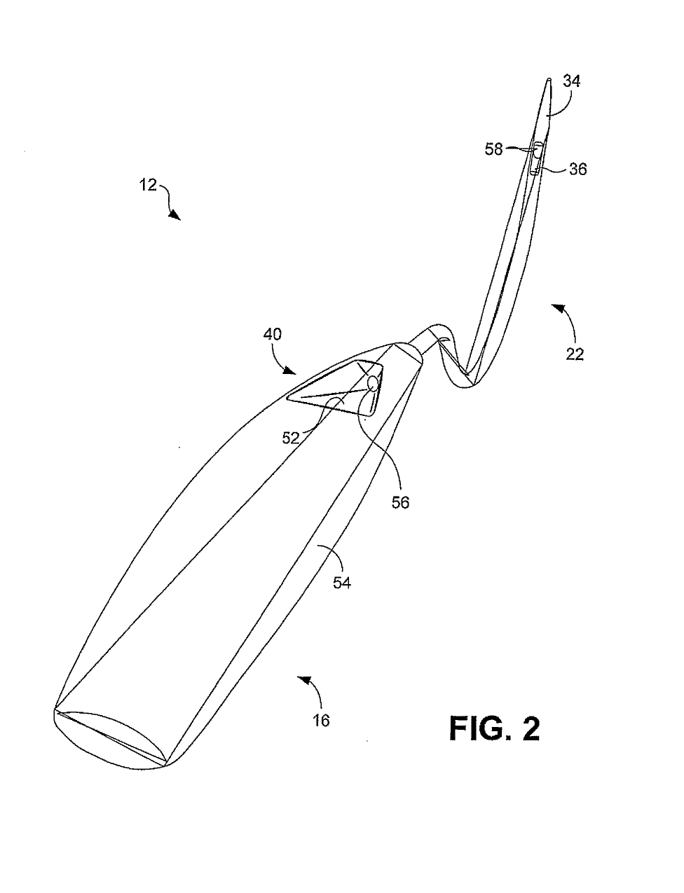

The needle 22 is hollow so as to form a cannula through which the snare 14 can be passed. More particularly, the needle 22 forms an inner lumen that extends from a first opening 36 of the needle to a second opening 38 of the needle. In the embodiment shown in FIG. 1, the first opening 36 is positioned adjacent the distal end 32 and the second opening 38 is positioned adjacent the proximal end 26. The second opening 38 is in open communication with a port 40 that is formed in the handle 16. As is described in greater detail below, the snare 14 can be passed through the port 40 and the second opening 38 to position the snare within the needle 22. The configuration of the port 40 is described in relation to FIG. 2.

In terms of materials, the handle 16 can be constructed of any suitable rigid material, such as a metal or a polymeric material. The needle 22 can be constructed of a biocompatibte, strong material, such as stainless steel. In some embodiments, the handle 16 and needle 22 can be composed of the same material and may even be unitarily formed together so as to have a monolithic configuration.

With continued reference to FIG. 1, the snare 14 comprises an elongated shaft 42 having a proximal end 44 and a distal end 46. The shaft 42 is flexible so as to enable the shaft to easily adept to the contours of the needle inner lumen and any body passages along which the snare is to travel. In some embodiments, the shaft 42 comprises a hollow tube through which a wire passes. In such cases, the shaft 42 can be constructed of a suitable flexible biocompatible material, such as a polymeric material. In other embodiments, the shaft 42 is solid and can be made of a polymeric material or a metal material, such as stainless steel or nitinol.

Provided at the proximal end 44 of the snare 14 is a grip element 48 that, as described below, is used to manipulate the snare relative to the introducer 12. Provided at the distal end 46 of the snare 14 is an implant coupling element 50 that is configured to couple to and secure an implant that is to be positioned with the body. In the illustrated embodiment, the coupling element 50 is formed as a loop. Such a loop can be formed from a flexible wire constructed of a polymeric or metal material. In such a case, the wire can extend from the gripping element 48, through the shaft 42, and terminate in a loop. In some embodiments, nitinol is suitable for the construction of the coupling element 50 due to nitinol's shape memory characteristics. In particular, when nitinol is used, the coupling element 50 can easily be compressed to pass through the needle inner lumen, but can readily spring back to its original shape (e.g., loop shape) after emerging from the needle 22. In some embodiments, the shaft 42 and the coupling element 50 comprise a unitarily-formed element, such as an elongated wire that extends from the gripping element 48 and terminates in a loop. In such cases, the shaft 42 need not comprise a tube.

FIG. 2 is a perspective view of the introducer 12. As indicated in that figure, the port 40 of the handle 16 is formed by one or more surfaces 52 that extend inwardly from an outer surface 54 of the handle to an orifice 56 that is aligned with the second opening 38 of the needle 22 (FIG. 1). As is also visible in FIG. 2 the needle 22 includes a snare-deflecting surface 58 positioned within the first opening 36 that urges the snare 14 (FIG. 1) out from the needle when the snare is pushed against the surface.

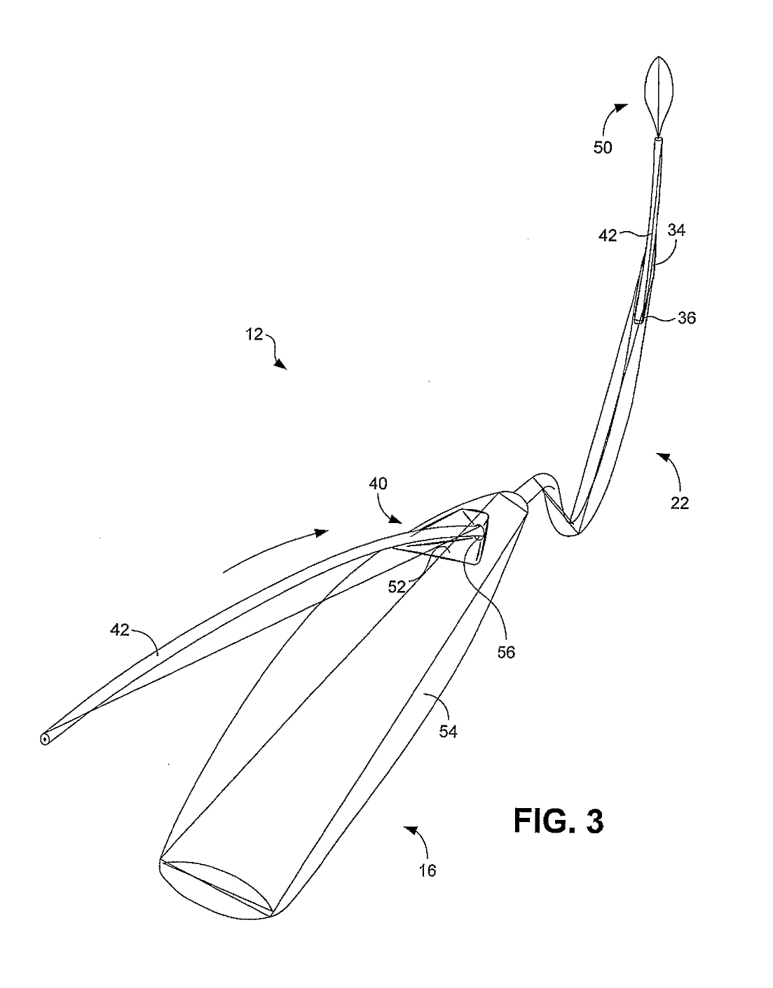

With the above-described system configuration, the snare 14 can be inserted through the port 40 and orifice 56 of the introducer handle 16, moved into the inner lumen of the introducer needle 22, pushed through the needle inner lumen, and made to exit the needle through the first opening 36. The result of that process is illustrated in FIG. 3.

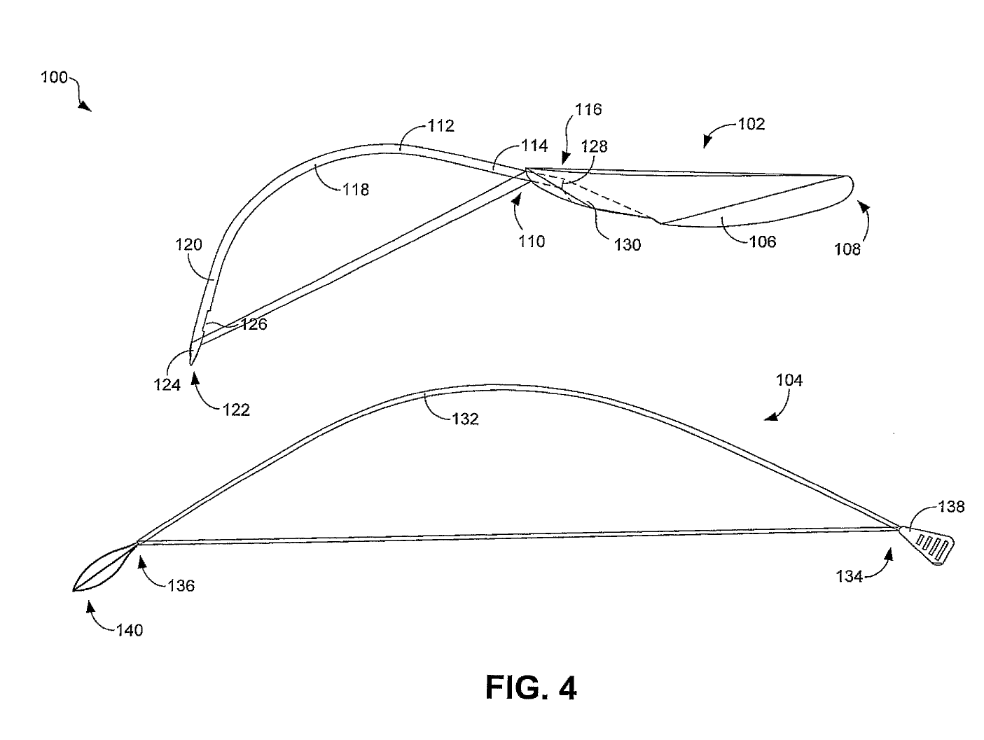

FIG. 4 illustrates a second embodiment of an introducer system 100. The introducer system 100 is similar to the system 10 described in relation to FIGS. 1-3, although the system 100 is configured for use in performing posterior prolapse repair and treating rectocele. As indicated in FIG. 4, the system 100 includes an introducer 102 and a snare 104. The introducer 102 comprises a handle 106 that includes a proximal end 108 and a distal end 110. The handle 106 is generally sized and shaped to fit within a surgeon's hand and, as depicted in FIG. 4, can be curved to facilitate firm gripping.

A needle 112 extends from the distal end 110 of the handle 102. As shown in FIG. 4, at least a substantial portion of the needle 112, like needle 22 (FIG. 1) is curved. In the embodiment of FIG. 4, however, the needle 112 is longer and straighter to enable passage of the needle deep into the pelvis. The needle 112 comprises a first generally straight portion 114 adjacent its proximal end 116, a curved portion 118 in a central region, and a second generally straight portion 120 adjacent its distal end 122. Formed at the distal end 122 is a blunt point or tip 124 that is configured to dissect soft tissue as the needle 112 is passed through the body.

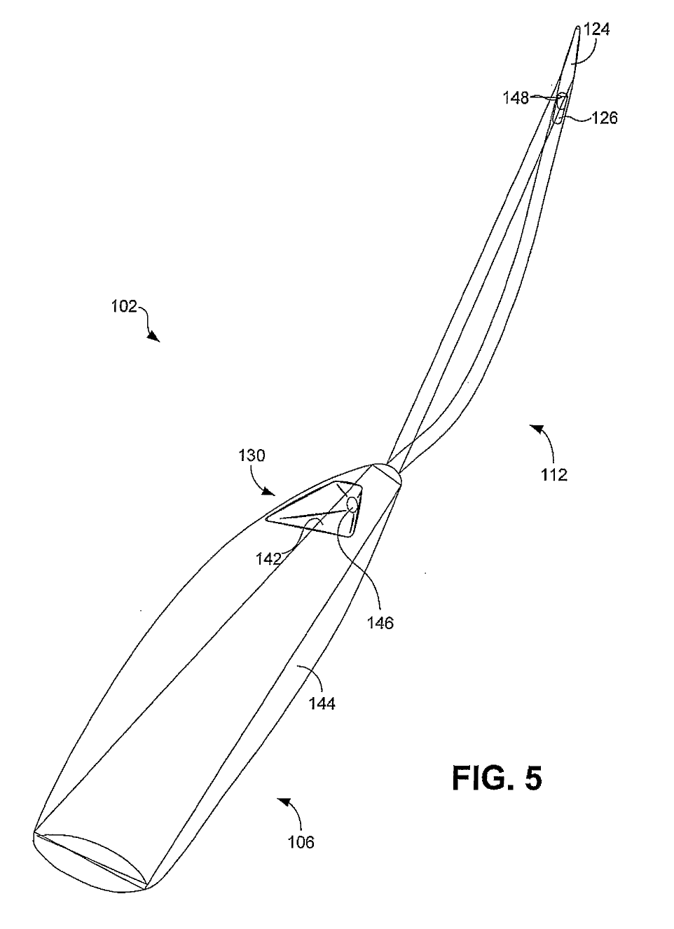

The needle 112 is hollow so as to form a cannula through which the snare 104 can be passed. More particularly, the needle 112 forms an inner lumen that extends from a first opening 126 of the needle to a second opening 128 of the needle. In the embodiment shown in FIG. 4, the first opening 126 is positioned adjacent the distal end 122 and the second opening 128 is positioned adjacent the proximal end 116. The second opening 128 is in open communication with a port 130 that is formed in the handle 106. As is described in greater detail below, the snare 104 can be passed through the port 130 and the second opening 128 to position the snare within the needle 112. The configuration of the port 130 is described in relation to FIG. 5.

In terms of materials, the handle 106 can be constructed of any suitable rigid material, such as a metal or a polymeric material. The needle 112 can be constructed of a biocompatible, strong material, such as stainless steel. In some embodiments, the handle 106 and needle 112 can be composed of the same material and may even be unitarily formed together so as to have a monolithic configuration.

With continued reference to FIG. 4, the snare 104 comprises an elongated shaft 132 having a proximal end 134 and a distal end 136. The shaft 132 is flexible so as to enable the shaft to easily adapt to the contours of the needle inner lumen and any body passages along which the snare is to travel. In some embodiments, the shaft 132 comprises a hollow tube through which a wire passes. In such cases, the shaft 132 can be constructed of a suitable flexible biocompatible material, such as a polymeric material. In other embodiments, the shaft 132 is solid and can be made of a polymeric material or a metal material, such as stainless steel or nitinol.

Provided at the proximal end 134 of the snare 104 is a grip element 138 that, as described below, is used to manipulate the snare relative to the introducer 102. Provided at the distal end 136 of the snare 104 is an implant coupling element 140 that is configured to couple to and secure an implant that is to be positioned with the body. In the illustrated embodiment, the coupling element 140 is formed as a loop. Such a loop can be formed from a flexible filament, such as a wire, constructed of a polymeric or metal material. In such a case, the wire can extend from the gripping element 138, through the shaft 132, and terminate in a loop. In some embodiments, nitinol is suitable for the construction of the coupling element 140 due to nitinol's shape memory characteristics. In particular, when nitinol is used, the coupling element 140 can easily be compressed to pass through the needle inner lumen, but can readily spring back to its original shape (e.g., loop shape) after emerging from the needle 112. In some embodiments, the shaft 132 and the coupling element 140 comprise a unitarily-formed element, such as an elongated wire that extends from the gripping element 138 and terminates in a loop. In such cases, the shaft 132 need not comprise a tube.

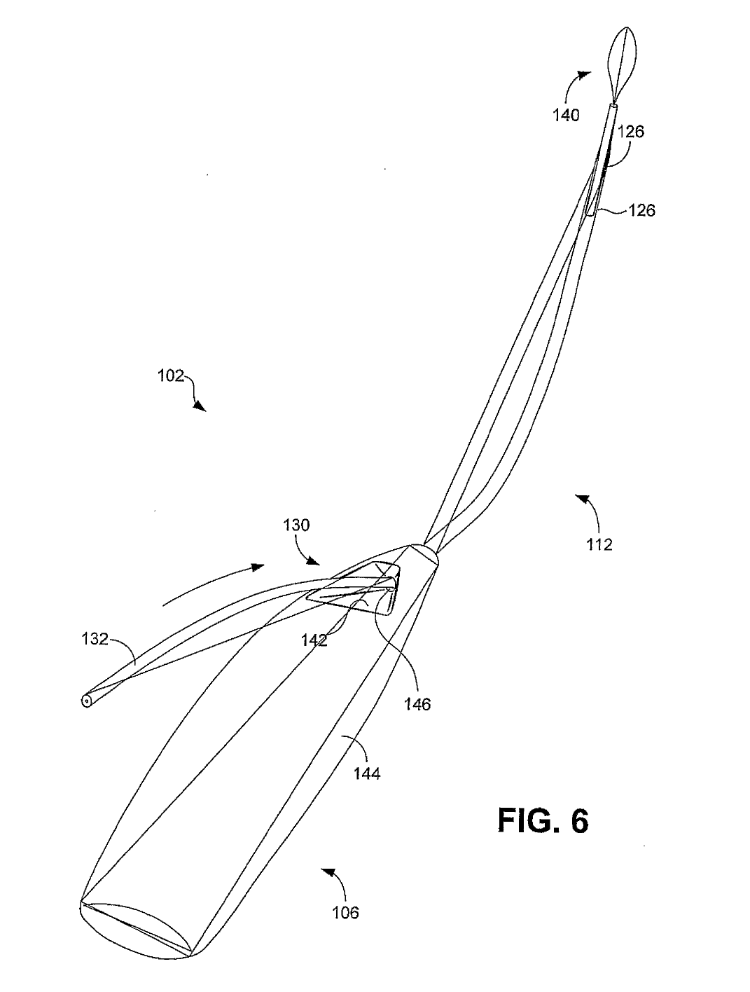

FIG. 5 is a perspective view of the introducer 102. As indicated in that figure, the port 130 of the handle 106 is formed by one or more surfaces 142 that extend inwardly from an outer surface 144 of the handle to an orifice 146 that is aligned with the second opening 128 of the needle 112 (FIG. 4). As is also visible in FIG. 6, the needle 112 includes a snare-deflecting surface 148 positioned within the first opening 126 that urges the snare 14 (FIG. 4) out from the needle when the snare is pushed against the surface.

With the above-described system configuration, the snare 104 can be inserted through the port 130 and orifice 146 of the introducer handle 106, moved into the inner lumen of the introducer needle 112, pushed through the needle inner lumen, and made to exit the needle through the first opening 126. The result of that process is illustrated in FIG. 6.

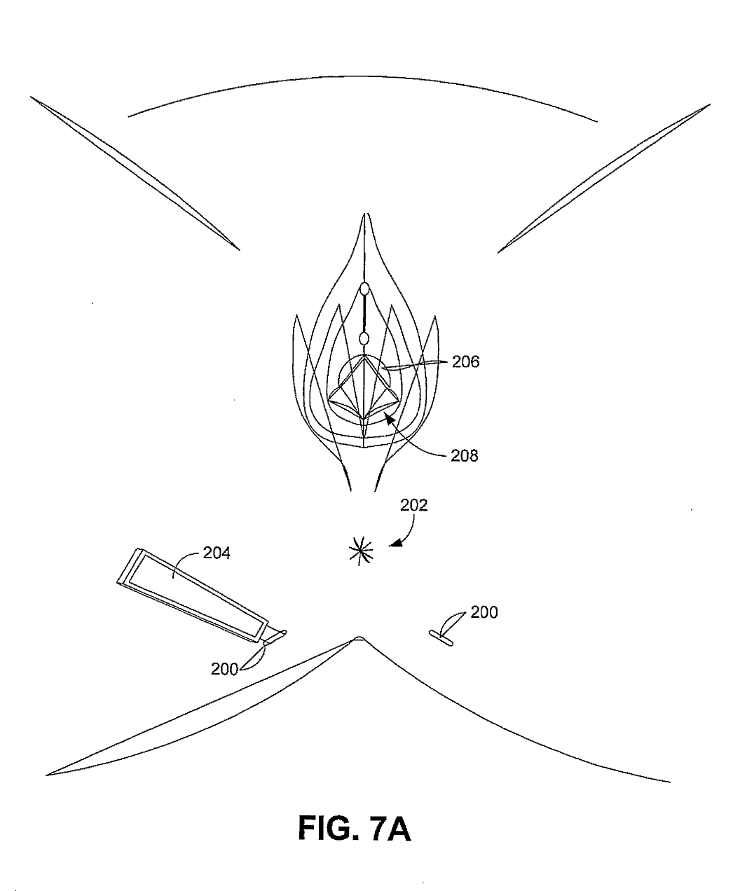

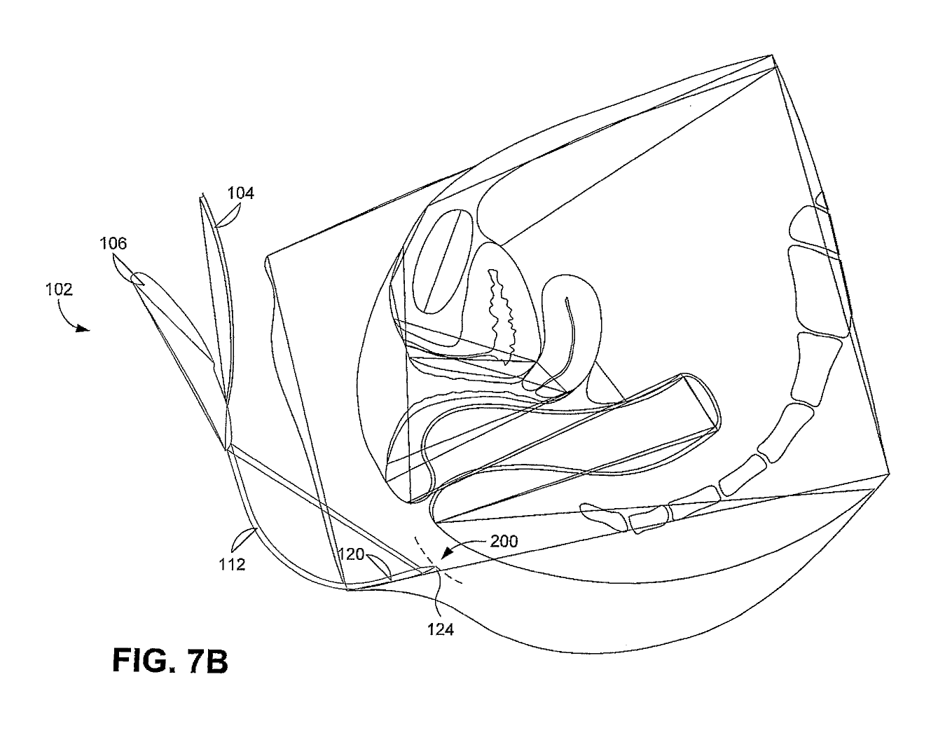

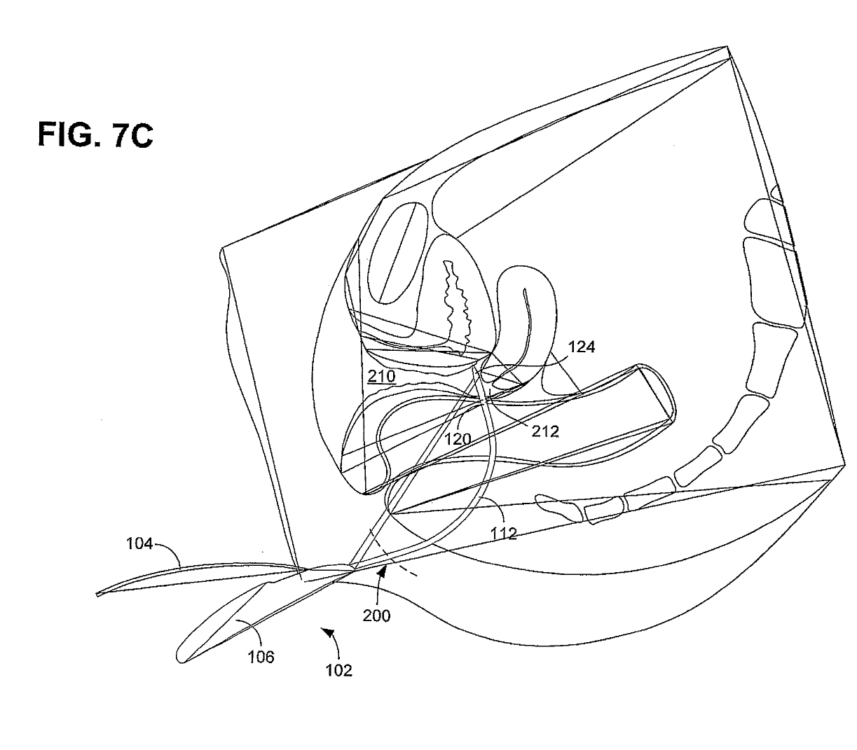

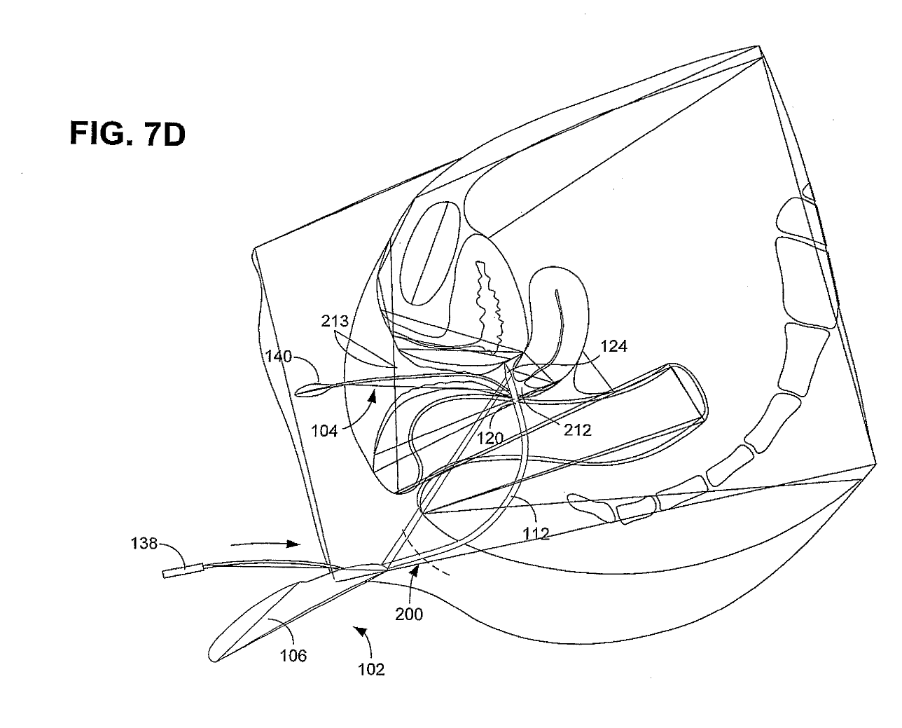

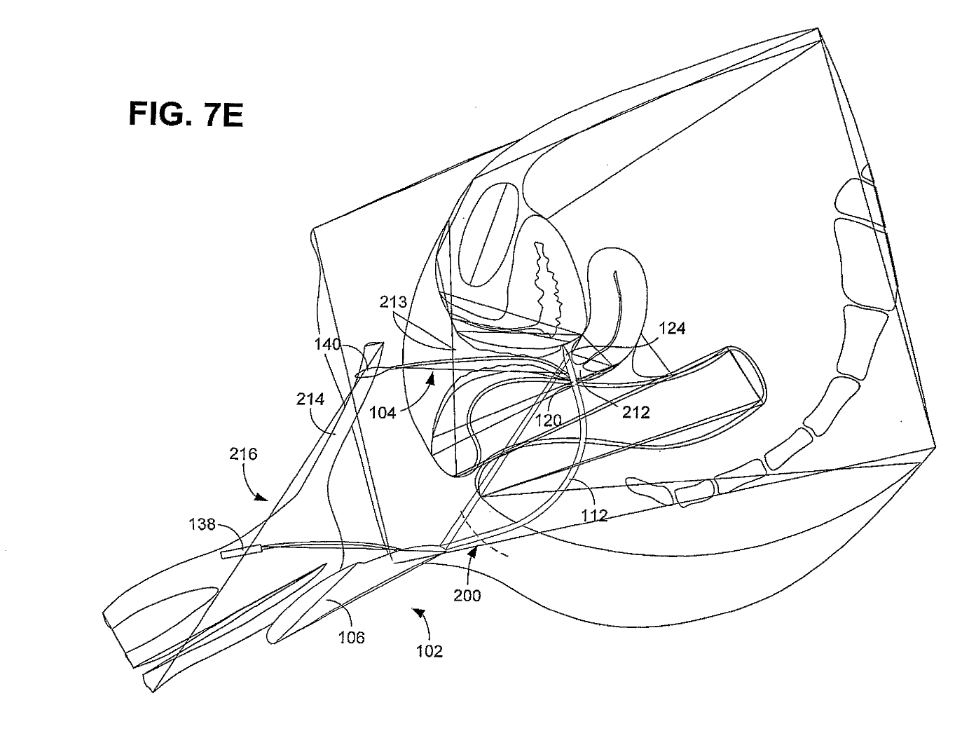

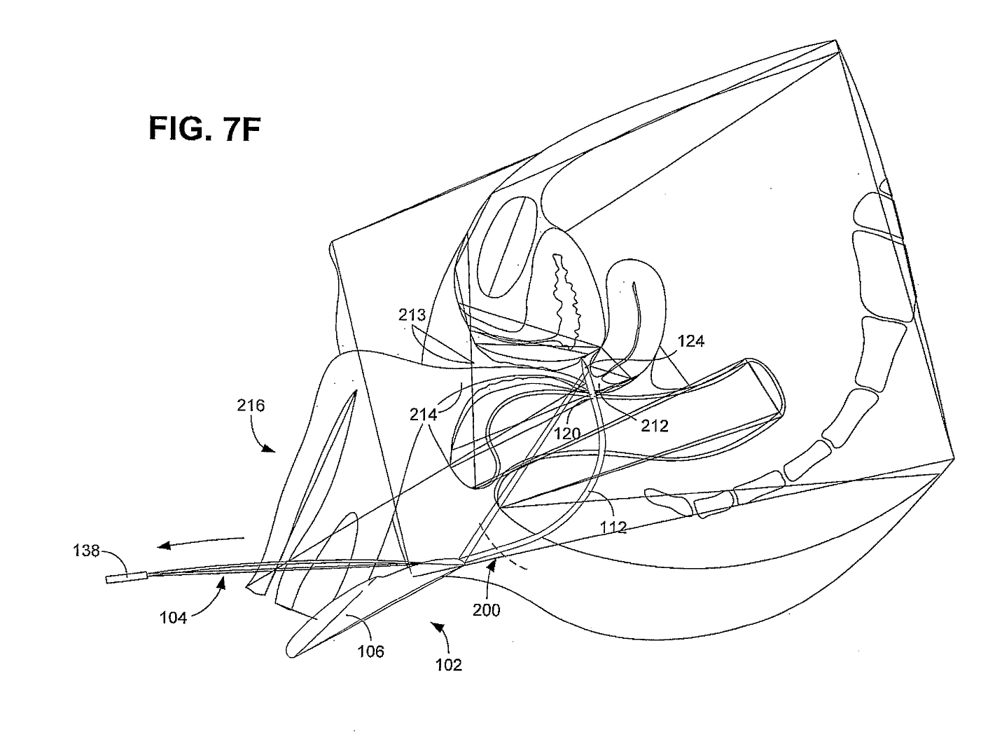

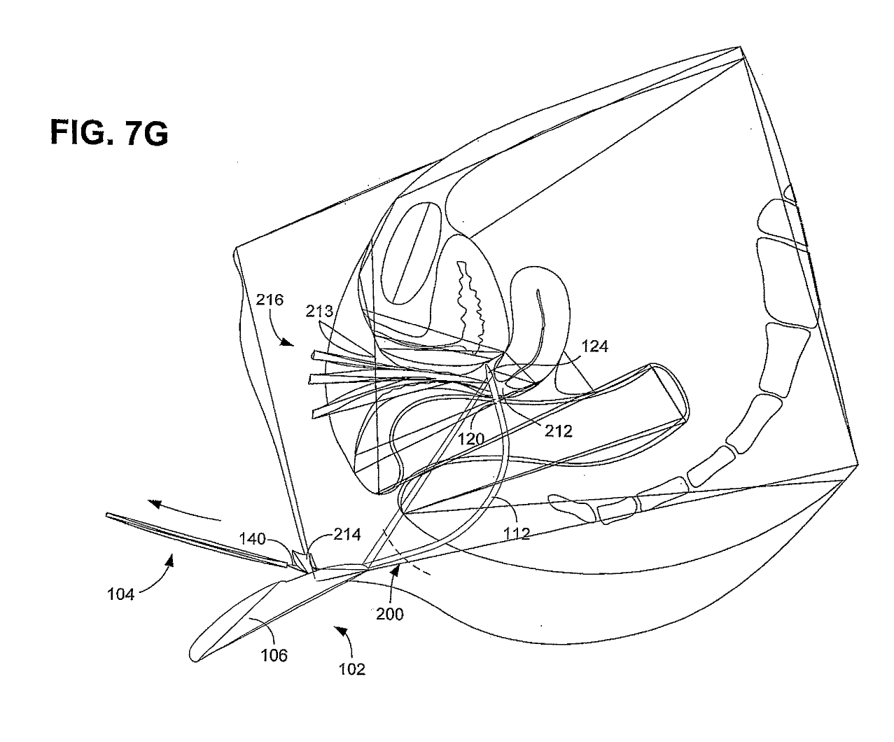

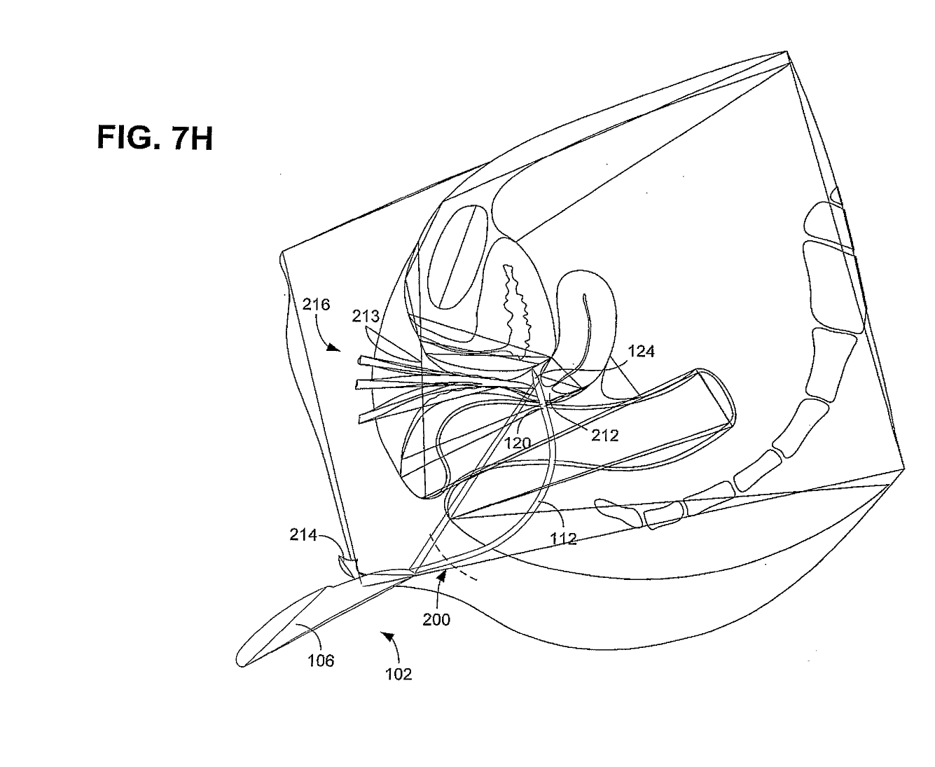

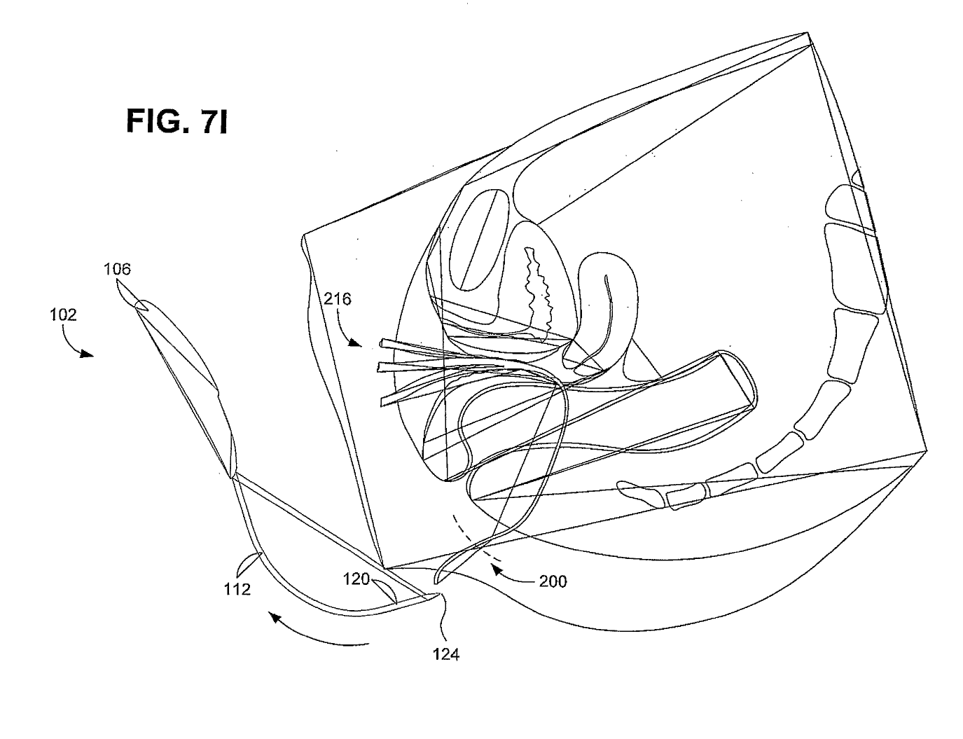

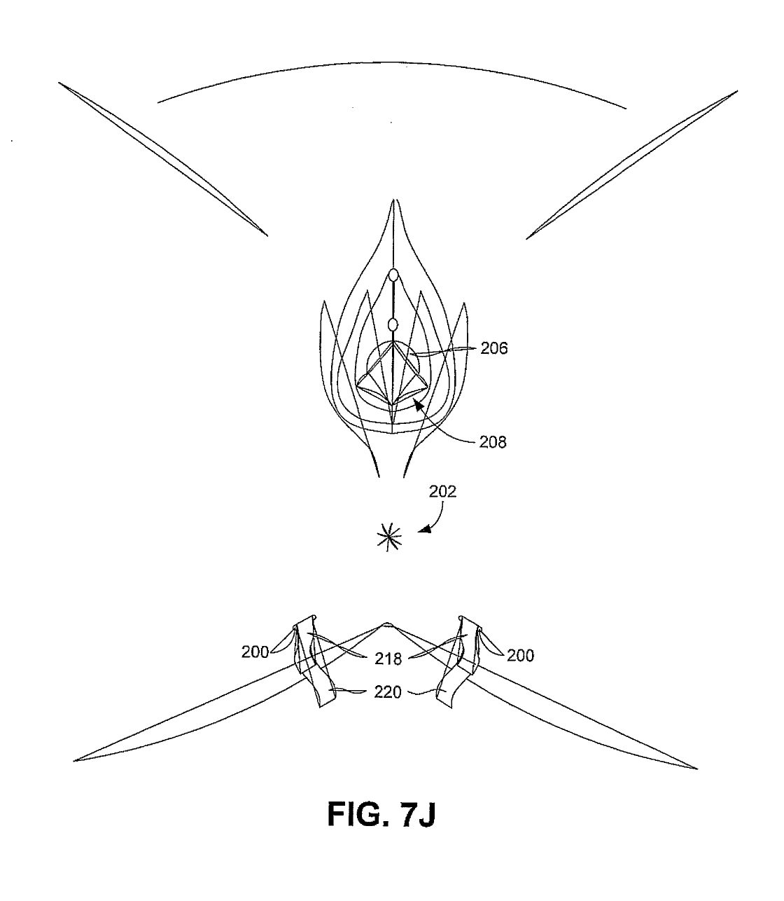

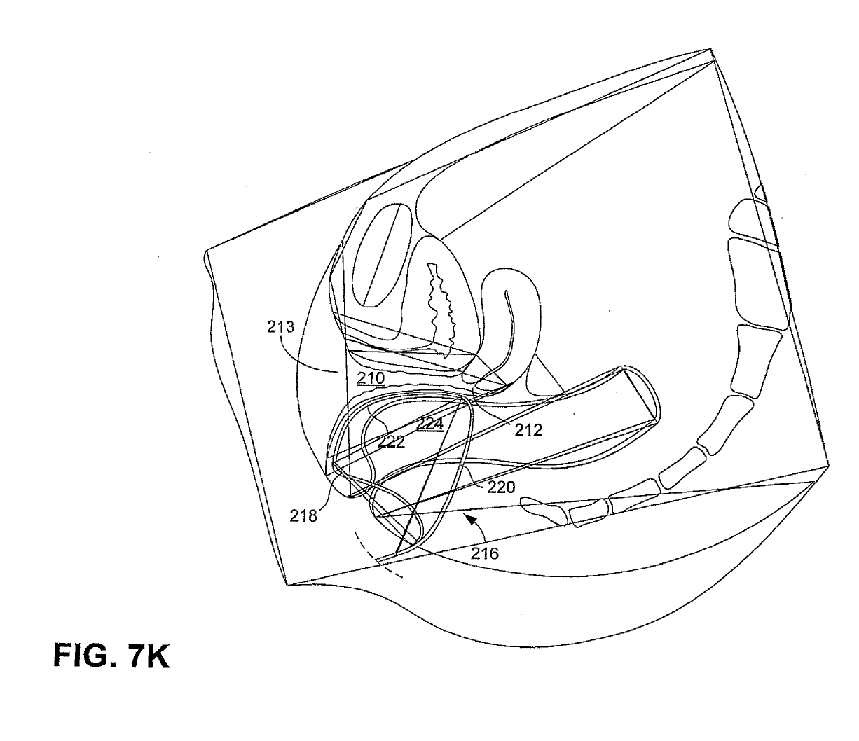

FIGS. 7A-7K illustrate a process for implanting an article using a system of the disclosure. More particularly, FIGS. 7A-7K illustrate a procedure for implanting a posterior prolapse repair implant between the vagina and the rectum using the introducer system 100 shown in FIG. 4. Although a posterior repair procedure is depicted in FIGS. 7A-7K and is described in detail in the following for purposes of describing the manner in which the disclosed introducer systems can be used to introduce an implant, it is to be understood that the procedure is described for purposes of example only. As stated above, similar systems may be used to implant other implants in other surgical procedures, such as anterior prolapse repair or treatment of urinary incontinence.

Beginning with FIG. 7A, small pararectal incisions 200 are made on either side of the anus 202 with a sharp device, such as a scalpel 204. By way of example, the incisions 200 are made 2-3 centimeters (cm) posterior and lateral to the anus 202. In addition, a midline incision is made in the posterior vaginal wall 206 to form an opening 208 that extends from the vaginal introitus to the vaginal apex to provide access to the space between the vagina and the rectum. The vaginal mucosa may then be dissected away from the rectum using blunt and/or sharp dissection.

Turning to FIG. 7B, the tip 124 of the introducer needle 112 is positioned at one of the incisions 200 with the introducer 102 oriented so that the handle 106 is substantially vertical and the second straight portion 120 of the needle is substantially parallel to the vagina 210. Referring next to FIG. 7C, the introducer needle 112 is passed through the incision 200 and through the soft tissue of the pelvis toward the ischial spine (not shown). As the needle 112 passes through the soft tissue, the introducer 102 is rotated so that the second straight portion 120 approaches a vertical orientation, as indicated in the figure. The needle tip 124 is advanced through the posterior vaginal wall and into the vaginal vault 212 such that the tip is positioned within the vagina. That process can be aided by placing a finger within the vagina to guide the needle tip 124 into position.

With reference to FIG. 7D, the snare 104, which can have been positioned already within the introducer 102 or later inserted therein is extended from a retracted position in which the implant coupling element 140 is contained within the inner lumen of introducer needle 112 to an extended position in which the coupling element extends beyond the first opening 126 (FIG. 4) of the needle. The snare 104 is then extended through the introducer 102, for example using the gripping element 138, until the implant coupling element 140 passes out from the vaginal introitus 213, as indicated in FIG. 79.

Referring next to FIG. 7E, a relatively long anchoring arm 214 of an implant 216 is coupled to the implant coupling element 140. By way of example, the implant 216 comprises a flexible mesh implant such that the arm 214 can be simply passed through the loop of the coupling element to secure the implant to the snare 104.

Turning to FIG. 7F, the snare 104 is retracted back into the introducer needle 112, for example using the gripping element 138, such that the implant coupling element 140 is again contained within the inner lumen of the needle. Due to the coupling between the implant 216 and the snare 104, a portion of the anchoring arm 214 may also be contained within the needle inner lumen. In some embodiments, a stop mechanism (not shown) can be provided within the needle inner lumen so as to limit the extent to which the snare 104 can be retracted into the needle inner lumen. For example, a stop (not shown), such as a bulbous portion, can be provided along the snare 104 adjacent the implant coupling element 140 that will abut a mating surface within the needle inner lumen, such as a constriction, adjacent the needle tip 124 so that the implant coupling element can be drawn into the needle inner lumen, but not farther through the needle inner lumen. Such a stop mechanism facilitates simultaneous withdrawal of the snare 104 and the needle 112. In other embodiments, the snare 104 need not be retracted back into the introducer needle 112 at all. In such an embodiment, the needle 112 and snare 104 can be withdrawn from the patient together with the snare in the extended position, if desired.

With reference next to FIG. 7G, at least a portion of the implant arm 214 can be pulled through the inner lumen of the introducer needle 112 so as to position the anchoring arm 214 in the passage that extends between the incision 200 and the vagina 210, which was formed by the needle. Notably, because the implant arm 214 is placed into that position while still contained within the needle 112, damage to the soft tissues in which the passage has been formed is reduced, as is the friction that resists such positioning. As shown in FIG. 7G, the snare 104 can be retracted to the point at which the implant coupling element 140 and the anchoring arm 214 exit the introducer handle 106. At that point, the anchoring arm 214 has been properly positioned within the body for subsequent adjustment, if necessary. As mentioned above, however, the snare 104 can, alternatively, be retracted to a limited extent due to the provision of a stop mechanism, or can not be retracted at all, as desired by the surgeon performing the procedure.

Assuming the snare 104 is retracted to the point at which it exits the introducer handle, the anchoring arm 214 is released from the implant coupling element 140, as indicated in FIG. 7H. Then, as indicated in FIG. 7I, the introducer needle 112 can be withdrawn from the body through the incision 200, thereby leaving the anchoring arm 214 in place within the tissues of the pelvis with a portion of the arm extending out from the incision. As mentioned above, the snare 104 can be withdrawn from the body simultaneous to withdrawal of the needle 112 in cases in which the snare is not withdrawn from the needle inner lumen (e.g., due to provision of a stop mechanism) or in cases in which the snare is not retracted back into the needle inner lumen after extension at all. The same result is achievable in such cases, however, given that the anchoring arm 214 can still be drawn through the passage formed by the needle 112 until a portion of the arm extends from the incision 200. The primary difference in such cases is that the anchoring arm 214 is in direct contact with the soft tissue of the passage as it passes through the passage instead of travelling through the needle inner lumen.

At this point: a similar procedure can be followed for positioning the opposite arm of the implant 216 using the other pararectal incision 200. That is, the introducer needle 112 can be passed through the incision 200 to the vaginal vault 212 on the opposite side of the vagina 210 and the opposite implant arm can be positioned in the passage formed by the needle. In addition the relatively short arms of the implant can be positioned in other passages extending from the incisions on opposite sides of the vagina 210 to a position adjacent the vaginal introitus 213. Once that has been completed, a portion of a relatively short arm 218 and a portion of a relatively long arm 220 extends out from each pararectal incision 200, as indicated in FIG. 7J, and a central body 222 (FIG. 7K) of the implant 216 can be positioned between the vagina 210 and the rectum 224 to provide a support structure that prevents encroachment of the rectum into the vaginal space. Finally, the implant arms 218, 220 can be appropriately tensioned, for example by pulling excess length out from the incisions 200, and the portions of the arms that extend outside of the body trimmed. The final result of the implantation is illustrated in FIG. 7K, with the implant body 222 positioned between the vagina 210 and the rectum 224.

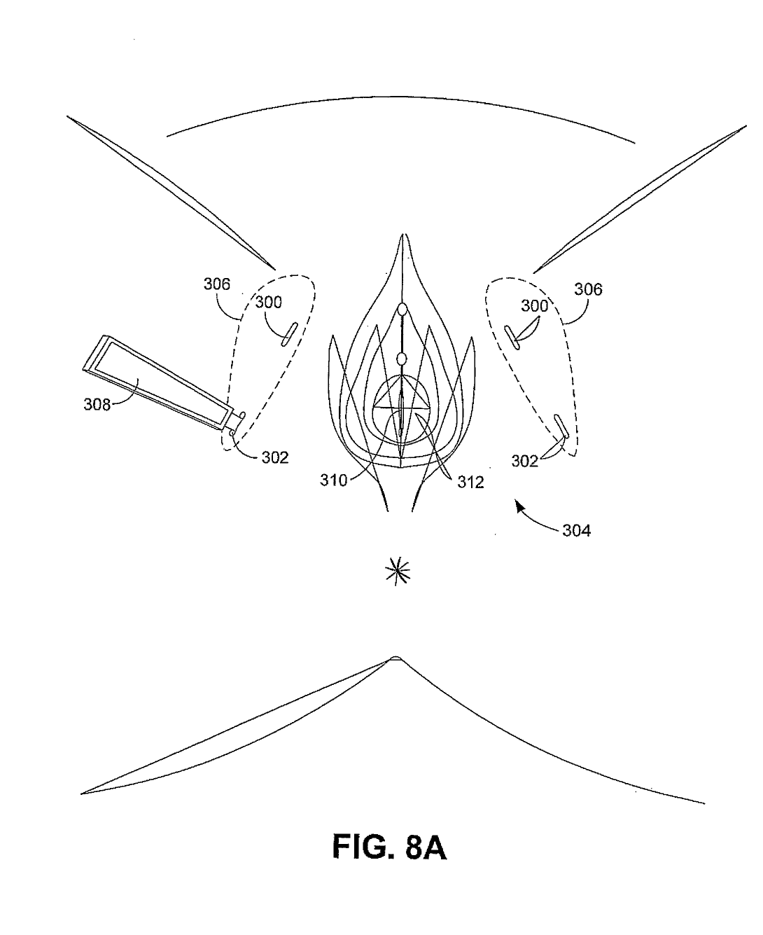

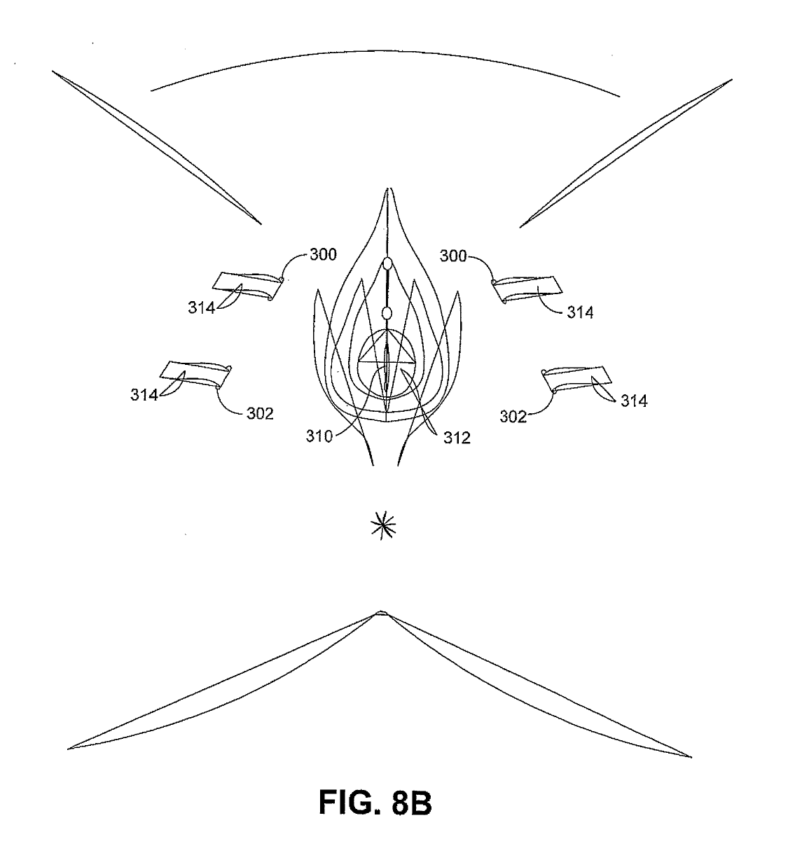

As described above, other implantation procedures can be performed using similar introducer systems. For example, anterior prolapse repair can be performed. To perform such a procedure, similar steps to those described above are completed. The primary differences include the shape of the implant, the location of the incisions made in the pelvis, and the positioning of the implant within the pelvis. As shown in FIG. 5A superior and inferior incisions 300 and 302 can be made in the paravaginal region 304 in alignment with the obturator foramina 306 of the pubic bone. Again, those incisions 300 and 302 can be made with a sharp device, such as a scalpel 308. In addition, a midline incision 310 can be made in the anterior vaginal wall 312 to provide access to the space between the vagina and the urethra. Each of four arms can be positioned within passages that extend from the incisions 300 and 302 to the vagina to position a body of the implant between the vagina and the urethra. As shown in FIG. 8B, portions 314 of the arms extend from the incisions 300 and 302 can then be trimmed as described above in relation to the posterior prolapse repair procedure.

As is also described above, the introducer systems can be used to treat urinary incontinence. In such a procedure, similar steps are performed except that the implant can comprise a urethral sling that is positioned below the urethra to provide support to the urethra. The ends of the sling can, for example, be passed through and/or embedded in the obturator foramina, or can be otherwise secured to hard or soft tissue of the pelvis.

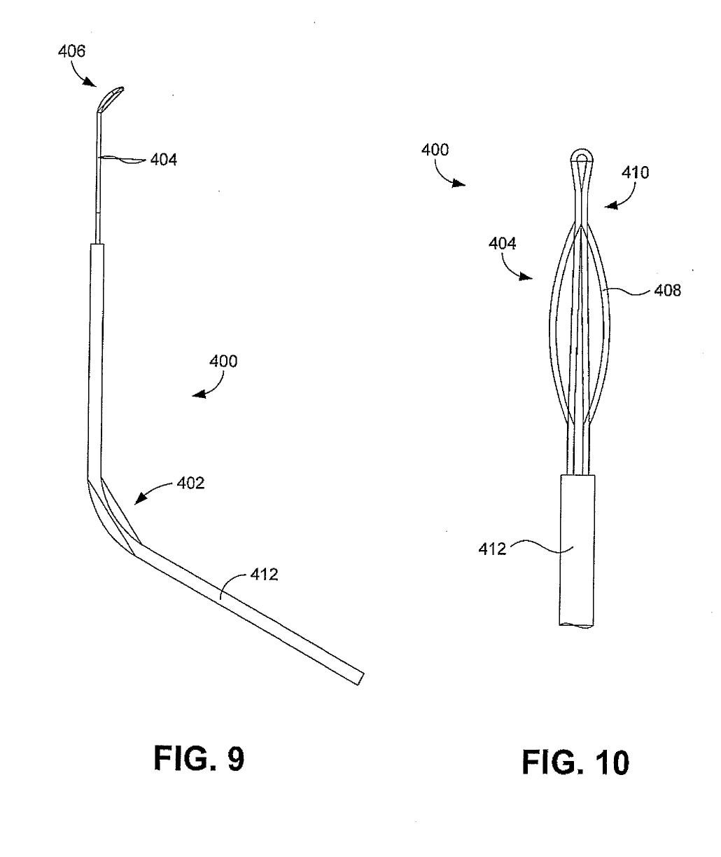

FIGS. 9 and 10 illustrate an alternative embodiment of a snare 400. Referring first to FIG. 9, the snare 400 can be formed as a wire constructed of a suitable metal material, such as stainless steel or nitinol. The snare 400 is pre-shaped to have a bend 402 that facilitates manipulation of the snare when positioned within the vagina or other body passage in which it is used. In particular, the bend 402 provides steering capability to the snare 400 so that the implant coupling element 404 of the snare can be moved in a desired direction, for example by twisting the snare using a grip element of the snare (not shown). As indicated in FIG. 9, the implant coupling element 404 comprises a further bend 406 that reduces the likelihood of snagging of the snare 400 within the vagina once the snare has been extended from its introducer needle.

Turning to FIG. 10, the implant coupling element 404 comprises a loop 408 and a constriction 410 that is: for example, positioned at a distal end of the loop. With such a configuration, an implant can be securely held by the implant coupling element 404 by first passing a portion of the implant through the loop 408 and then passing the implant portion into the constriction 410: such that the implant is securely clamped by the constriction. As is apparent from FIG. 10, the implant coupling element 404 can be formed from a wire that extends from a shaft 412 and forms the loop 408 and the constriction 410. In an alternative arrangement, the snare 400 can only comprise one or more wires that form the loop 408 and constriction 410, as well as the shaft.

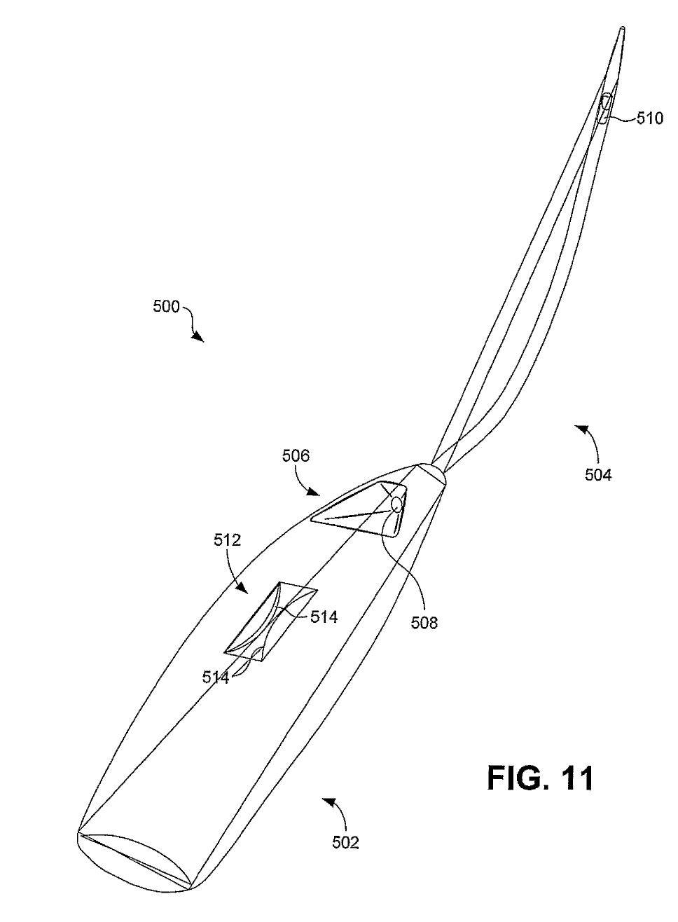

FIG. 11 illustrates a further embodiment of an introducer 500. As indicated in that figure, the introducer 500 comprises a handle 502 and a needle 504. As with the previously-described embodiments, the handle includes a port 506 that defines an orifice 508, which leads to an inner lumen of the needle 504. The needle 504 includes an opening 510 in communication with the inner lumen that enables a snare to be extended from the needle. Unlike the previously-described embodiments, however, the introducer 500 includes a cleat 512 comprising opposing inner surfaces 514 that are adapted to secure a snare relative to the introducer such that snare is positioned in a desired position along the inner lumen of the needle 504 when so secured.

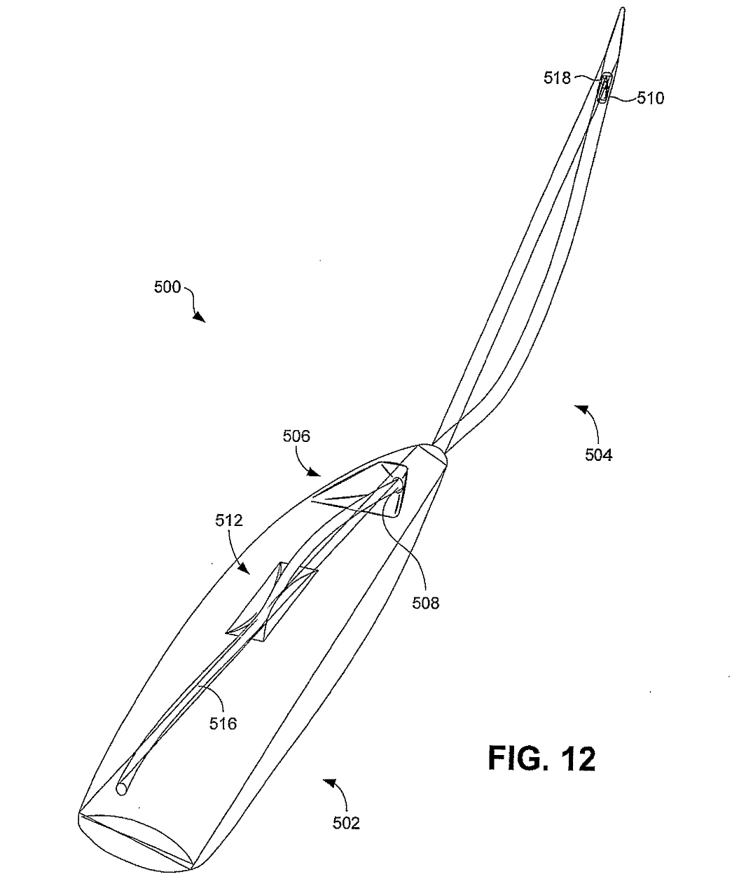

Turning to FIG. 12, securing of a snare 516 with the introducer 500 is depicted, As shown in that figure, the snare 516 is pushed into the cleat 512 such that the snare is securely clamped by the opposing inner surfaces 514 of the cleat. In some embodiments, the snare 516 can comprise indicia (not shown) that indicate what portion of the snare is to be secured within the cleat 512 such that indexing is provided as to important positions of the snare within the introducer 500. For example, indicia can be provided on the snare 516 at a position that, when aligned with the cleat, correspond to a position at which an implant coupling element 518 of the snare is positioned just within the opening 510 of the needle 504. In other embodiments, the snare 516 can comprise a complementary feature (not shown), such as mating indentations or protuberances, that are specifically adapted to interface with the cleat 512.

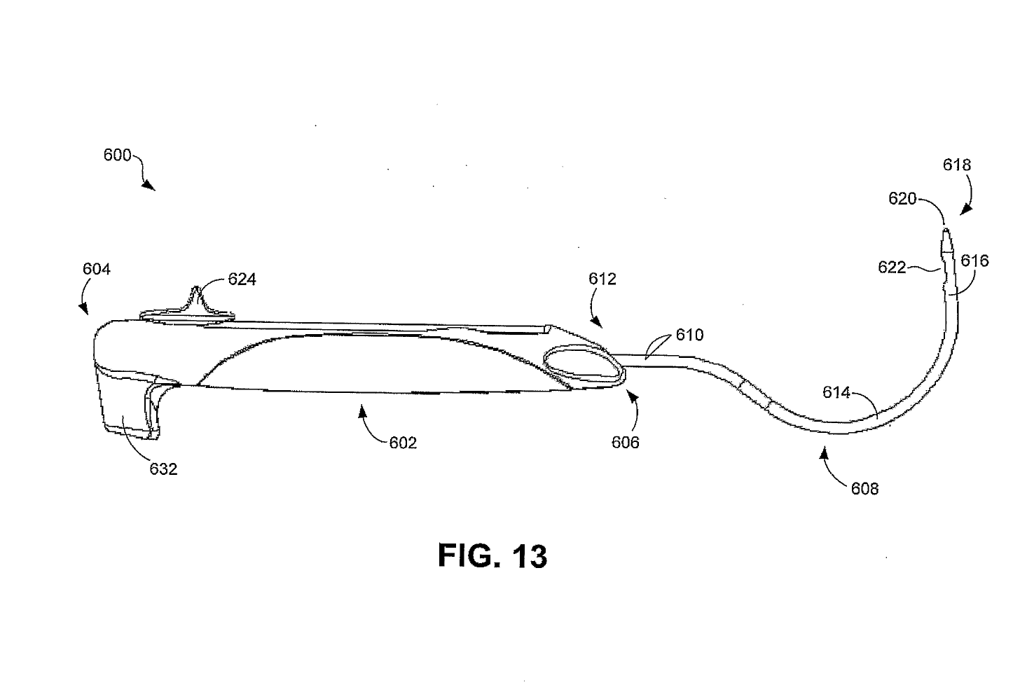

FIGS. 13-17 illustrate another embodiment of an introducer 600. The introducer 600 is well suited for use in performing prolapse repair, such as anterior prolapse repair, and treating cystocele. As indicated in FIG. 13: the introducer 600 comprises a handle 602 having a proximal end 604 and a distal end 606. The handle 602 is generally sized and shaped to fit within a surgeon's hand.

A needle 608 extends from the distal end 606 of the handle 602. At least a portion of the needle 608 is curved. In the embodiment of FIG. 13, the needle 608 comprises a first generally straight portion 610 adjacent its proximal end 612: a curved portion 614 in a central region, and a second generally straight portion 616 adjacent its distal end 618. Formed at the distal end 618 is a blunt point or tip 620 that is configured to dissect soft tissue as the needle 608 is passed through the body.

The needle 608 is hollow so as to form a cannula in which an internal snare (not visible in FIG. 13) can be positioned. More particularly, the needle 608 forms an inner lumen that extends from a first opening (not visible in FIG. 13) of the needle to a second opening 622 of the needle. In the embodiment shown in FIG. 13, the second opening 622 is positioned adjacent the distal end 618 of the needle 608. As described in greater detail below, the internal snare can be extended out from the second opening 622 to couple an implant to the snare.

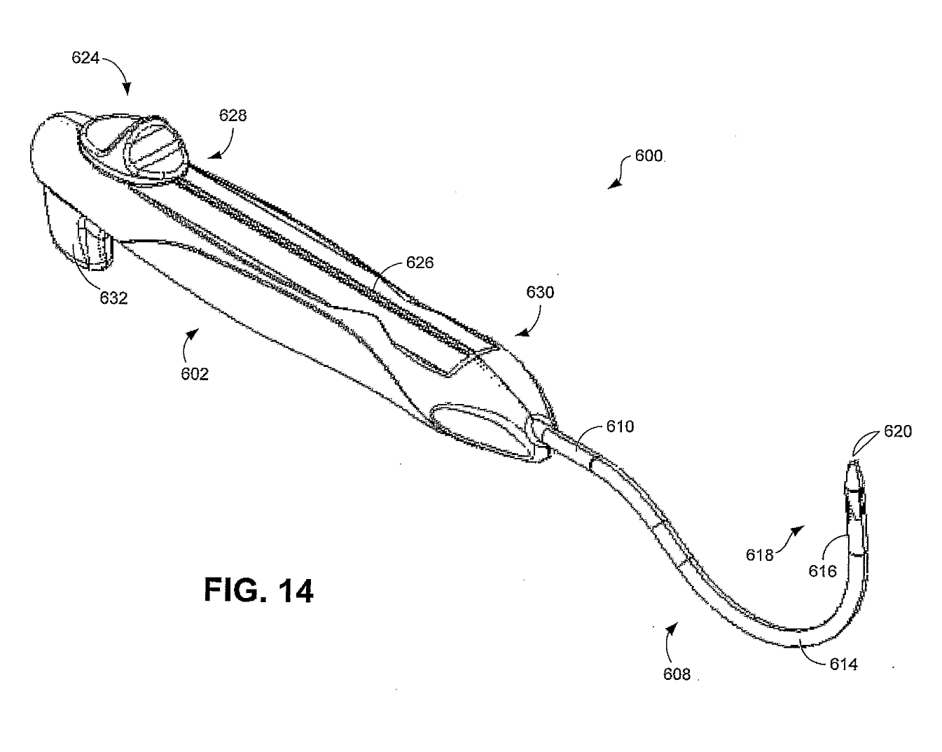

Extension, and retraction, of the internal snare is controlled with a snare extension mechanism that includes an external slide element 624 that is provided on the handle 602. As indicated in FIG. 14, the slide element 624 is positioned within an elongated slot 626 provided along the length of the handle 602. In the retracted orientation illustrated in FIGS. 13 and 14, the slide element 624 is positioned at a proximal end 628 of the slot 626. In the fully-extended position (not shown) the slide element 624 is positioned at a distal end 630 of the slot.

Further provided on the handle 602 is a gripping protrusion 632 that assists the surgeon in gripping and controlling the introducer 600 and, as described below, may enable the surgeon to displace the slide element 624 along the slot 626 in the distal direction with a single hand.

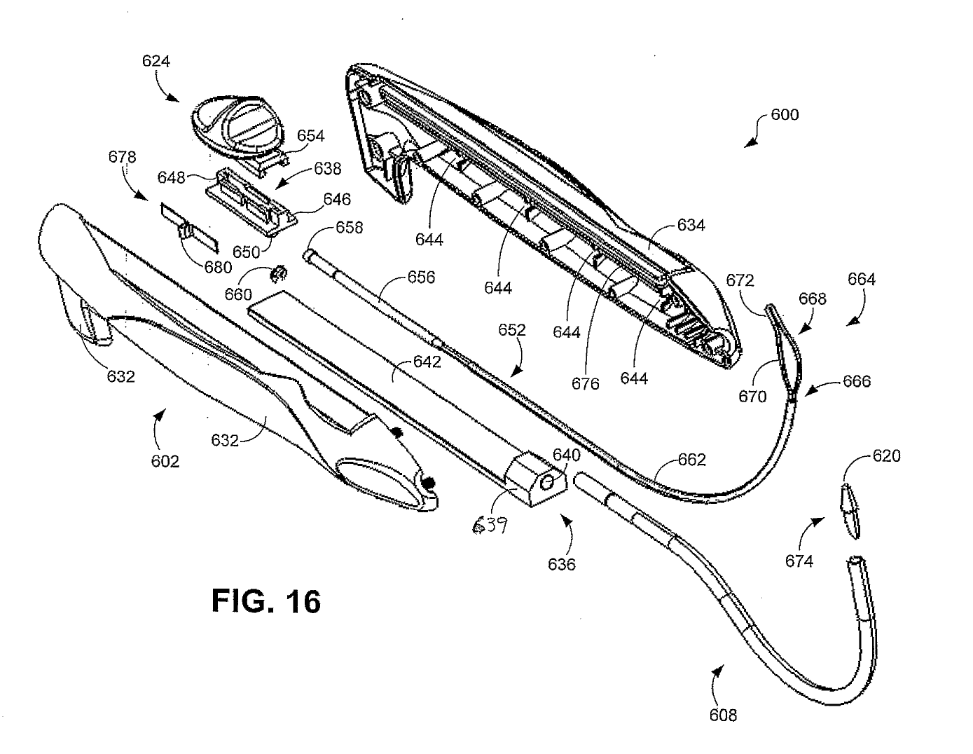

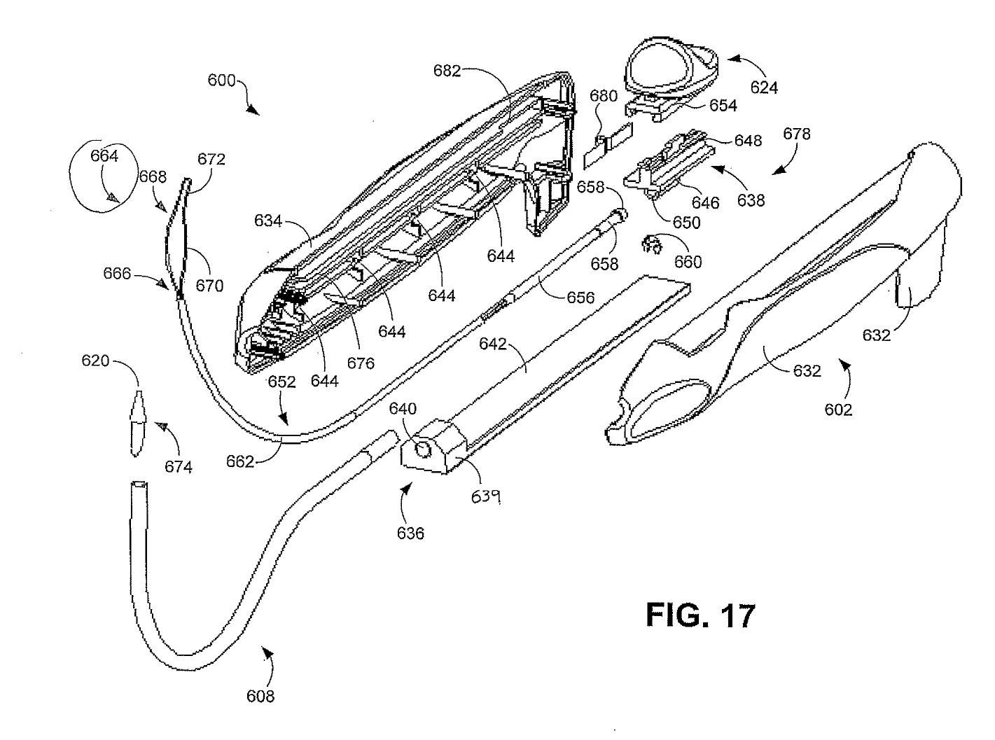

FIGS. 16 and 17 illustrate the introducer 600 in a disassembled or “exploded” state. As indicated in those figures, the handle 602 is formed from two opposed, mating portions 632 and 634. By way of example, the mating portions 632, 634 are formed from a semi-rigid polymeric material. In such a case, the mating portions 632, 634 can, for example, be formed through an injection molding process. Disposed within an interior space defined by the two portions 632, 634 are a needle support member 636 to which the needle 608 mounts and a carrier element 638 to which the slide element 624 mounts.

The needle support member 636 is formed from a strong, hard materials such as stainless steel, and serves to distribute stresses that may be imposed upon the handle 602 by the needle 608 when the introducer 600 is used in a surgical procedure. In the embodiment of FIGS. 16 and 17, the needle support member 636 comprises a coupling portion 639 having an opening 640 adapted to receive the needle 608. Once disposed within the opening 640, the needle 608 an, for example: be welded to the needle support member 636. The needle support member 636 further comprises an elongated tang 642 that extends prosimally from the coupling portion 639. Provided within each of the mating portions 632, 634 of the handle 602 are notches 644 that are adapted to receive the tang 642 when the introducer 600 is assembled.

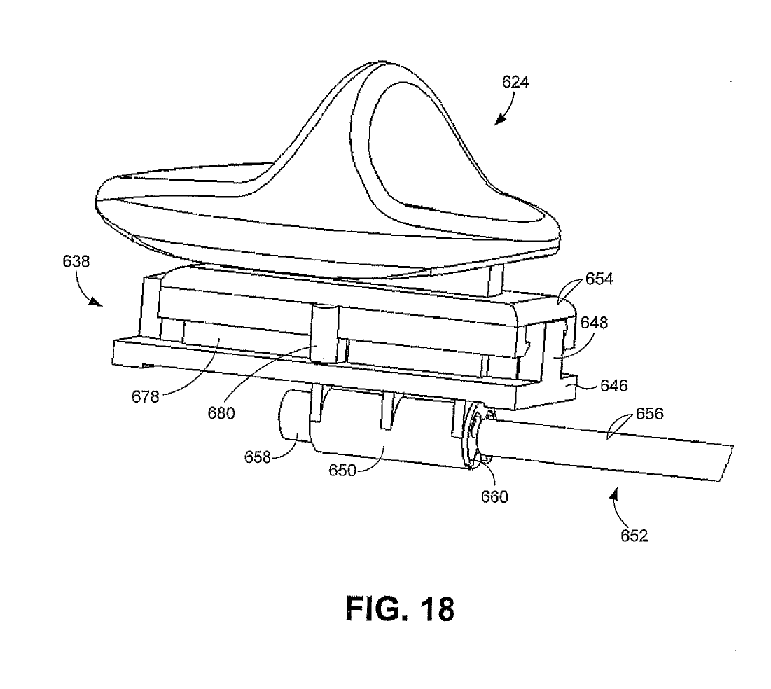

The carrier element 638 generally comprises a base 646, a slide element coupling portion 648, and a snare coupling portion 650. As indicated in FIG. 18, which illustrates the carrier element 638 coupled to both the slide element 624 and to the internal snare 652, the coupling portion 648 of the carrier element is received within a complimentary coupling portion 654 of the slide element. In some embodiments, the coupling portion 648 snap fits within the coupling portion 654 such that the slide element 624 and the carrier member 638 can move together as one integrated part along the slot 626 (FIG. 14).

As further illustrated in FIG. 18, the snare coupling portion 650 can be configured as a cylindrical element in which a rigid shaft 656 of the snare 652 can be positioned. The snare 652 can be so positioned by threading the snare through the snare coupling portion 650 until a head 658 of the shaft 656 abuts against the snare coupling portion 650 as shown in FIG. 18. In some embodiments, a retainer clip 660 is used to maintain the position of the shaft 652 relative to the carrier element 638.

Returning to FIGS. 16 and 17, the internal snare 652 includes not only the rigid shaft 656 but also a flexible portion that can be extended through and, at least partially, out from the needle 608. In the embodiment of FIGS. 16 and 17, the flexible portion comprises a flexible sheath 662 that partially surrounds a flexible wire 664. By way of example, the sheath 662 is composed of a polymeric material, such as polytetrafluoroethylene (PTFE), and the wire 664 is composed of a metal material, such as stainless steel or nitinol. The wire 664 extends from the shaft 656 to a point beyond the distal end 666 of the sheath 662. At the distal end 666 the wire 664 forms an implant coupling element 668 having a configuration similar to that described in relation to FIG. 10. Specifically, the implant coupling element 668 comprises a loop 670 and a constriction 672 that is, for example, positioned at a distal end of the loop. With such a configuration, an implant can be securely held by the implant coupling element 668 by first passing a portion of the implant through the loop 670 and then passing the implant portion into the constriction 672.

As is further illustrated in FIGS. 16 and 17, the tip 620 of the needle 608 can be provided on a tip member 674 that is manufactured separately from the remainder of the needle. In such a case, the tip member 674 can be fixedly secured to the needle 608, for example by welding, after both components have been separately formed.

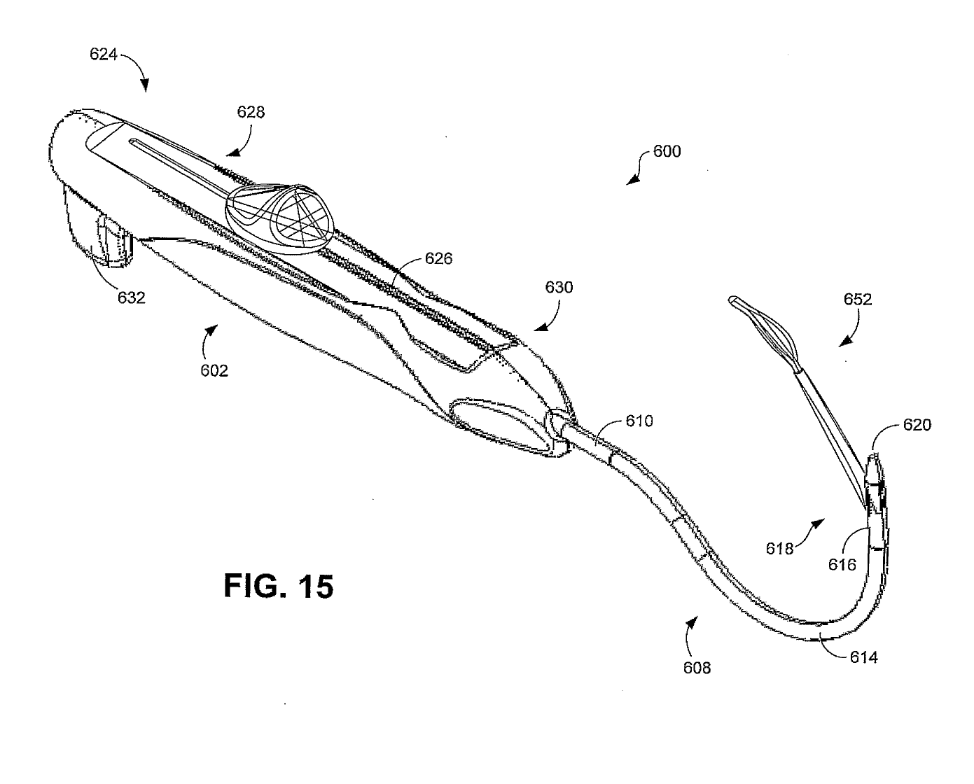

With the various internal components described above in relation to FIGS. 16-18 the internal snare 652 can be extended out from the needle 608 after the needle tip 620 has been positioned in an appropriate location within the patient's body, for instance within the vagina. To accomplish such extension, the slide element 624 is displaced toward the distal direction (i.e., toward the distal end 606 of the handle 602). In some cases, the snare 652 can be extended using a single hand. Specifically, the gripping protrusion 632 can be gripped using the index finger and the slide element 624 can be simultaneously pushed forward along the slot 626. The surgeon can then “walk” his or her hand up along the handle 602 to enable further pushing of the slide element 624 with the thumb. When the slide element 624 is moved distally along the slot 626, the carrier element 638 is likewise moved and the lateral edges of the carrier element base 646 slide within guide slots 676 provided within the mating portions 632, 634 of the handle 602 (see FIGS. 16 and 17). FIG. 15 depicts the snare 652 in an extended orientation that results from displacement of the slide element 624 and its associated carrier element 638.

In some embodiments, the introducer 600 further comprises a locking and/or indexing feature that incorporates an internal biasing element 678. As described in greater detail below, the locking/indexing feature prevents unintended extension of the internal snare 652 as well as provides feedback to the surgeon to indicate when a particular retracted position has been attained. In the embodiment shown in FIGS. 16-18, the biasing element 678 comprises a narrow strip of metal, such as stainless steel, that has been bent to form a nodule 680. The biasing element 678 is adapted to be partially encapsulated by the slide element 624 and the carrier element 638 with the nodule 680 extending laterally outward, as indicated in FIG. 18. When the internal snare 652 is at or near the fully-retracted position, the nodule 680 is positioned within a space defined by a notch 682 formed in mating portion 632, as indicated in FIG. 17. Given that the notch 682 does not extend along the entire length of the guide slot 676 of the mating portion 632, however, the nodule 680 is compressed when the slide element 624 is displaced to extend the internal snare 652.

The functionality described above serves two purposes. First, because the nodule 680 must be compressed to move the slide element 624 beyond a predetermined retracted position, a relatively large force is required to initiate snare extension and, therefore, the potential for unintentional extension of the snare 652 is reduced. After the snare 652 has been extended by the surgeon to connect an implant to the snare and then retracted to pull the implant adjacent the needle tip 620, the biasing element 678 provides an audible and/or tactile indication to the surgeon as to the position of the snare. Specifically, once the nodule 680 passes back into the notch 682, the nodule snaps back to its original, extended position, thereby providing positive indication to the surgeon as to how far the snare 652 has been retracted. In some embodiments, the notch 682 is configured such that further retraction is possible after the nodule 680 has snapped back into place within the notch 682. By way of example, the snare 652 is completely surrounded by the needle 608 when the slide element 624 is in the fully retracted position and only the implant coupling element 668 (or a portion thereof) extends out from the needle 608 when the slide element 624 is displaced such that the biasing element nodule 680 is positioned at the distal end of the notch 682 (i.e., the transition point between the notch and the remainder of the guide slot 676).

The introducer 600 described in relation to FIGS. 13-18 can be used to implant an article, such as an anterior prolapse repair implant, in similar manner to that described above in relation to FIGS. 8A and 8B. In particular, superior and inferior incisions can be made in the paravaginal region in alignment with the obturator foramina of the pubic bone. In addition, a midline incision can be made in the anterior vaginal wall to provide access to the space between the vagina and the urethra. Each of several arms of the implant can be positioned within passages that extend from the incisions to the vagina to position a body of the implant between the vagina and the urethra. To do that, the introducer 600 can be passed through the incisions and into the vagina with the blunt tip of the introducer dissecting the soft tissue between the incision and the vagina. Once the tip of the introducer has been positioned within the vagina, the internal snare of the introducer can be extended using the slide element (see FIG. 15) to facilitate attachment of an arm of the implant to the snare. Once the arm is so attached, the snare can be retracted, for example until the above-described feedback is registered, and then the needle can be pulled back through the passage formed by the needle dissection until the end of the implant arm extends from its incision in the manner shown in FIG. 88. Therefore, the procedure practiced with the introducer 600 is similar to that practiced with the introducer system 10 of FIG. 1 except that the snare does not comprise a separate component and is not withdrawn within the needle to the extent at which a major portion of the implant arm is placed within the needle prior to withdrawing the needle from the patient. Instead, the snare comprises an integral part of the introducer 600 and is retractable to a point at which the end of the implant arm is positioned adjacent or just within the distal end of the needle.

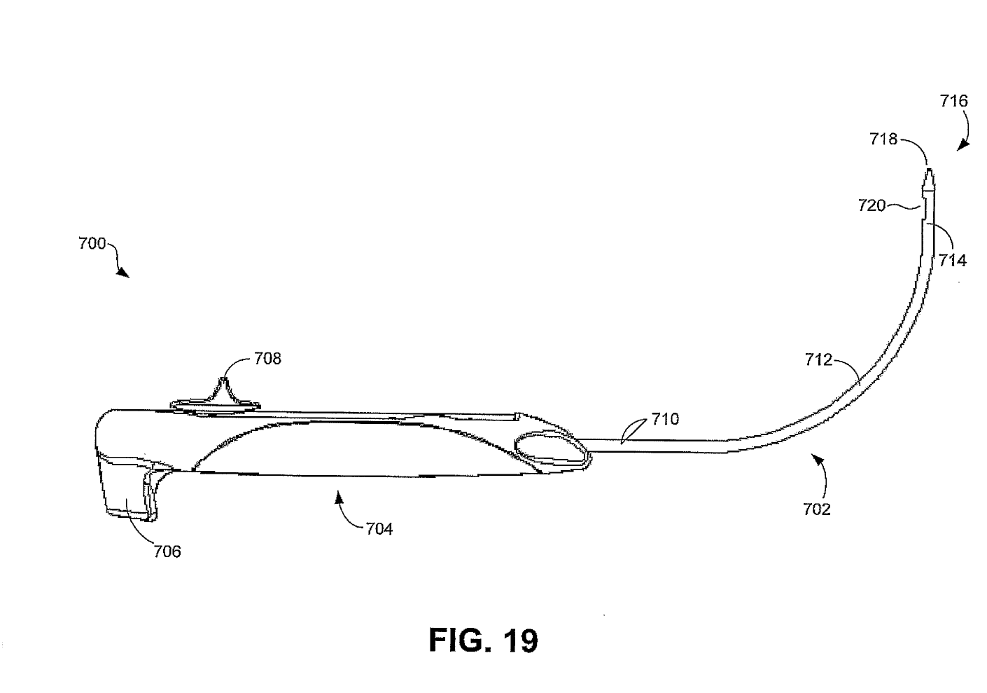

FIG. 19 illustrates an introducer 700 that is similar in many ways to the introducer 600, but that is dimensioned so as to be well suited for use in performing posterior prolapse repair and treating rectocele. The introducer 700 differs from the introducer 600 primarily in terms of the shape and dimensions of the needle 702 and the length of the internal snare (not shown) that can be extended from the needle. Accordingly, the introducer 700 also includes a handle 704 that comprises a gripping protrusion 706 and various internal components similar to those illustrated in FIGS. 16 and 17, which are controlled with a slide element 708. Like the needle 608, the needle 702 includes a first generally straight portion 710, a curved portion 712, and a second generally straight portion 714. Formed at the distal end 716 of the needle 702 is a blunt point or tip 718 that is configured to dissect soft tissue as the needle is passed through the body. The needle 702 is hollow so as to form a cannula in which the internal snare can be positioned. More particularly, the needle 702 forms an inner lumen having an opening 720 positioned adjacent the distal end 716 of the needle 702 from which the internal snare can be extended.

The introducer 700 can be used in surgical procedures similar to that described in relation to FIGS. 7A-7K with the most notable exceptions being that the snare does not comprise a separate component and is not withdrawn within the needle to the extent at which a major portion of the implant arm is placed within the needle prior to withdrawal of the needle from the patient. Instead, the snare comprises an integral part of the introducer 700 and is retractable to a point at which the end of the implant arm is positioned adjacent or just within the distal end of the needle.

1. An introducer comprising:
a handle;
a needle extending from the handle, the needle having a distal end and defining an inner lumen, the needle further having an opening that provides access to the inner lumen;
a snare having an implant coupling element, the snare being positioned within the inner lumen of the needle, the snare being extendable from the needle opening to an extended position in which the implant coupling element is positioned outside of the inner lumen and retractable to a retracted position in which at least a portion of the implant coupling element is positioned within the inner lumen; and
a snare extension mechanism provided on the handle, the extension mechanism being configured to alternately extend and retract the snare.

2. The introducer of claim 1, wherein the needle opening is positioned adjacent the distal end of the needle.

3. The introducer of claim 1, wherein the needle distal end forms a tip configured to dissect tissue as the needle is passed into the body through an external incision.

4. The introducer of claim 1, wherein the needle is sized and shaped such that the distal end can be positioned within the vagina when the needle is advanced through an external incision located in a paravaginal region or pararectal tissue.

5. The introducer of claim 1, wherein the implant coupling element comprises a loop configured to receive a portion of an implant.

6. The introducer of claim 5, wherein the loop is at least partially constructed of nitinol.

7. The introducer of claim 5, wherein the loop includes a constriction configured to securely clamp the implant.

8. The introducer of claim 5, wherein the snare comprises a tube and wherein the loop extends from the tube.

9. The introducer of claim 1, wherein the snare extension mechanism comprises an external slide element adapted to be displaced along a slot formed in the handle.

10. The introducer of claim 9, wherein the snare extension mechanism further comprises an internal carrier element to which the slide element is coupled, the carrier element being coupled to the snare.

11. The introducer of claim 1, wherein the snare extension mechanism comprises a feature that provides feedback to a surgeon indicating when a particular retracted position of the snare has been attained.

12. The introducer of claim 11, wherein the feature further prevents unintended extension of the internal snare.

13. The introducer of claim 11, wherein the feature comprises a biasing element that snaps into a notch provided in the handle when the snare has been retracted to the particular retracted position, the snapping action creating at least one of audible feedback or tactile feedback.

14. An implant introducer comprising:
an elongated and curved needle having a distal end, the needle further having an opening positioned adjacent the distal end and defining an inner lumen that extends to the opening, the needle further having a tip formed at the distal end, the tip being configured to dissect tissue as the needle is passed into the body through an external incision;
a handle from which the needle extends;
an elongated snare positioned within the needle, the snare having an implant coupling element at its distal end and being sized and shaped to be extended from the needle through the opening to an extended position at which the implant coupling element is positioned outside of the inner lumen such that an implant can be coupled to the implant coupling element; and
a snare extension mechanism provided on the handle, the extension mechanism being configured to alternately extend and retract the snare, the extension mechanism including an external slide element positioned within an elongated slot formed in the handle and an internal carrier element to which the external slide element is coupled, the carrier element being coupled to the snare such that when the slide element is displaced along the slide in a distal direction the snare is extended and when the slide element is displaced along the slide in a proximal direction the snare is retracted.

15. The introducer of claim 14, wherein the needle is sized and shaped such that the distal end can be positioned within the vagina when the needle has been advanced through the external pelvic incision.

16. The introducer of claim 14, wherein the implant coupling element comprises a loop configured to receive a portion of an implant.

17. The introducer of claim 16, wherein the loop includes a constriction configured to securely clamp the implant.

18. A method for positioning an implant within the body using an introducer, the method comprising:
passing a distal tip of a needle of the introducer through an external incision to a desired internal location,
extending a snare from the needle using a snare extension mechanism provided on a handle of the introducer;
connecting the implant to the snare;
retracting the snare back into the needle using the snare extension mechanism; and
withdrawing the needle from the body with the snare positioned adjacent or within the needle so as to draw the implant through a passage formed by the needle when the needle is passed from the external incision to the desired internal location.

19. The method of claim 18, wherein passing a distal tip of a needle comprises passing the needle distal tip through a paravaginal region or through pararectal tissue and into the vagina.

20. A method for positioning a mesh implant within the body, the method comprising:
passing a distal tip of an introducer needle through an external pelvic incision into the vagina, the needle comprising an inner lumen;
extending an elongated snare from an opening adjacent the needle distal tip to the vaginal introitus by operating a snare extension mechanism provided on a handle of the introducer;
passing an arm of the mesh implant through a loop formed at a distal end of the extended snare;
retracting the snare back into the needle by operating the snare extension in mechanism in a retraction direction until at least a portion of the loop is retracted back into the needle inner lumen; and
withdrawing the needle from the body so as to draw the implant through a passage formed by the needle when the needle is passed from the external incision to the vagina until a portion of the mesh implant arm extends out from the external incision.
